(19) United States
(12) Patent Application Publication
(10) Pub. No.: US 2006/0207978 A1
(43) Pub. Date: Sep. 21, 2006

(54) Tactile feedback laser system

(76) Inventors: Peter R. Rizun, Calgary (CA); Garnette R. Sutherland, Calgary (CA)

Correspondence Address: LAW OFFICE OF MARC D. MACHTINGER, LTD., 750 W. LAKE COOK ROAD, SUITE 350, BUFFALO GROVE, IL, 60089, US

(21) Appl. No.: 11/261,445

(22) Filed: Oct. 28, 2005

Related U.S. Application Data
(60) Provisional application No. 60/622,603, filed on Oct. 28, 2004.
(60) Provisional application No. 60/650,508, filed on Feb. 8, 2005.

(51) Int. Cl. B23K 26/02; B23K 26/16; B23K 26/14; A61B 18/18

(52) U.S. Cl. 219/121.83; 606/10; 219/121.67; 606/13

(57) ABSTRACT

A robot surgical laser with haptic feedback. The device allows an operator to feel surfaces using only light, and synthesize haptic feedback through a robot arm held by the operator when the focal point of the laser is coincident with a real surface, giving the operator the impression of touching something solid.

### CROSS-REFERENCE TO RELATED APPLICATIONS

This application claims the benefit under 35 USC 119(e) of provisional application No. 60/622,603 filed Oct. 28, 2004 and provisional application No. 60/650,508 filed Feb. 8, 2005.

### BACKGROUND OF THE INVENTION

When a surgeon makes an incision with a knife, there is instant feedback that indicates when contact is made with a surface and applied force. When a surgeon operates with a laser, there is no feedback. The surgeon is missing a sense of touch. Without the sense of touch, the surgeon must rely on sight and experience, possibly compromising dexterity and limiting surgical outcome. This invention is designed to address this limitation in laser surgery, and also has application in other laser cutting applications.

### SUMMARY OF THE INVENTION

There is therefore provided according to an aspect of the invention, a tactile feedback system comprising a robot arm, a remote distance measuring device mounted on the robot arm, the remote distance measuring device having an output corresponding to a distance measure, a hand control for the robot arm, and a control system responsive to the distance measure to adjust force applied to the hand control. The hand control may be a part of the robot arm, or may be a separate device with its own actuator. In a further aspect of the invention, the tactile feedback system includes a cutting laser, for example a surgical laser, mounted on the robot arm. In a further aspect of the invention, the control system is configured to adjust cutting laser power output depending on the distance measure. In further aspects of the invention, the force applied to the hand control increases non-linearly with proximity to a surface sensed by the remote distance measuring device or the force applied to the hand control depends on the motion of the robot arm. In a further aspect of the invention, the remote distance measuring device comprises a second laser. In a further aspect of the invention, the remote distance measuring device is configured to determine distance based on spot size of a beam emitted by the second laser and incident on a surface. In a further aspect of the invention, the hand control is physically remote from the robot arm.

These and other aspects of the invention are set out in the claims, which are incorporated here by reference.

### BRIEF DESCRIPTION OF THE FIGURES.

Preferred embodiments of the invention will now be described with reference to the figures, in which like reference characters denote like elements, by way of example, and in which:

FIG. 1 is a schematic of a tactile feedback laser system according to the invention;

FIG. 2 is a schematic of a distance measuring system according to the invention;

FIG. 3 are graphs showing resolution of an ambiguity in distance measurement using the system of FIG. 2;

FIG. 4 is a schematic showing force vectors from the deformation of a virtual surface;

FIG. 5 is an equation describing the feedback force from deformation of a virtual surface;

FIG. 6 is an equation describing laser intensity as a function of applied force;

FIG. 7 is a flow diagram illustrating basic method steps for the algorithm used to operate the distance measuring system of FIG. 2; and

FIGS. 8 and 9 illustrate respectively how the laser tracks a surface and how laser intensity increases with downward force.

### DETAILED DESCRIPTION OF PREFERRED EMBODIMENTS

In the claims, the word “comprising” is used in its inclusive sense and does not exclude other elements being present. The indefinite article “a” before a claim feature does not exclude more than one of the feature being present.

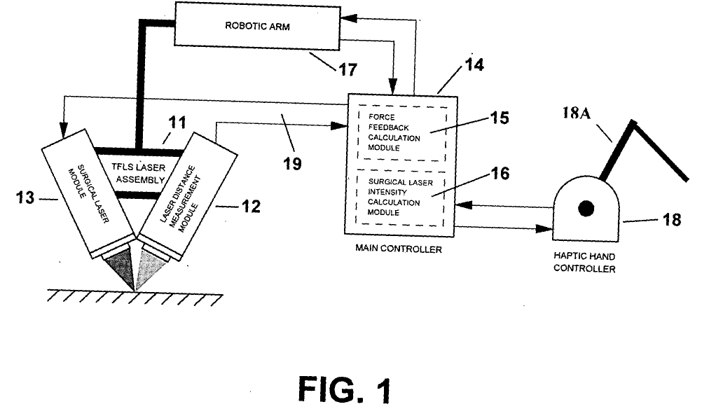

A tactile feedback system 11, shown for example in FIG. 1, includes a remote distance measuring device 12 mounted on the robot arm 17, a hand control 18 for the robot arm 17, an actuator 18A for the hand control 18, and a control system 14 responsive to the distance measure to adjust force applied by the actuator 18A on the hand control 18. The actuator 18A and hand control 18 form part of a haptic feedback system. Haptic devices, which provide tactile sensation to humans interacting with computers, are well known. The tactile feedback system 11 described here is a laser system (TLFS) that synthesizes haptic feedback when the focal point of the laser is coincident with a real surface, giving the operator the impression of touching something solid. This virtual surface felt by the operator will possess stiffness and frictional properties that change dynamically in response to sensor readings. Although nothing but light ever contacts the real surface, the operator will receive information about its properties through haptic channels. When applied to laser surgery, the TFLS controls cutting intensity in response to operator-applied force. Just as a knife penetrates to a greater depth with additional pressure, the haptic surgical laser could ablate more quickly with increased force.

The robot arm 17 may be a haptic-enabled, master-slave surgical robot system such as neuroArm™ as describe in United States patent publication No. 2004/0111183, the content of which is hereby incorporated by reference.

The TFLS incorporates the TFLS laser assembly 11 and software running on the surgical robot's main control system 14. The TLFS laser assembly 11 comprises a laser distance measurement system 12 that measures the distance between the focal point of its internal laser 20 and the point on the surface along the axis of the laser 20 and a surgical cutting laser 13. The software running on main control system 14 can be thought of as two modules: a software module 15 that determines how to render tactile feedback at hand controller 18 based on the laser distance measurement as well as position and velocity information from robot arm 17 and software module 16 that controls surgical laser intensity based on force.

The TFLS uses the following aspects of the master-slave surgical robot: The laser distance measurement system 12 and surgical laser 13 are attached as the end-effector of robot arm 17. The software modules for rendering tactile feedback and controlling surgical laser intensity run inside main control system 14 of the surgical robot. The software modules have access to robot arm 17 kinematics information such as arm position and velocity, through main control system. 14. Tactile-feedback is rendered through the robot workstation haptic hand control 18. Electrical interface 19 on the robot facilitates communication of distance information from the laser measurement system 12 to the main control system 14, and allows laser intensity to be controlled from the main control system 14. Certain parts of these devices, such as surgical lasers, robot arms and haptic hand controllers are known in the art and need not be described in great detail. The communication links between the devices that are represented as lines are also conventional components used in computer systems and may be wireless or wired links.

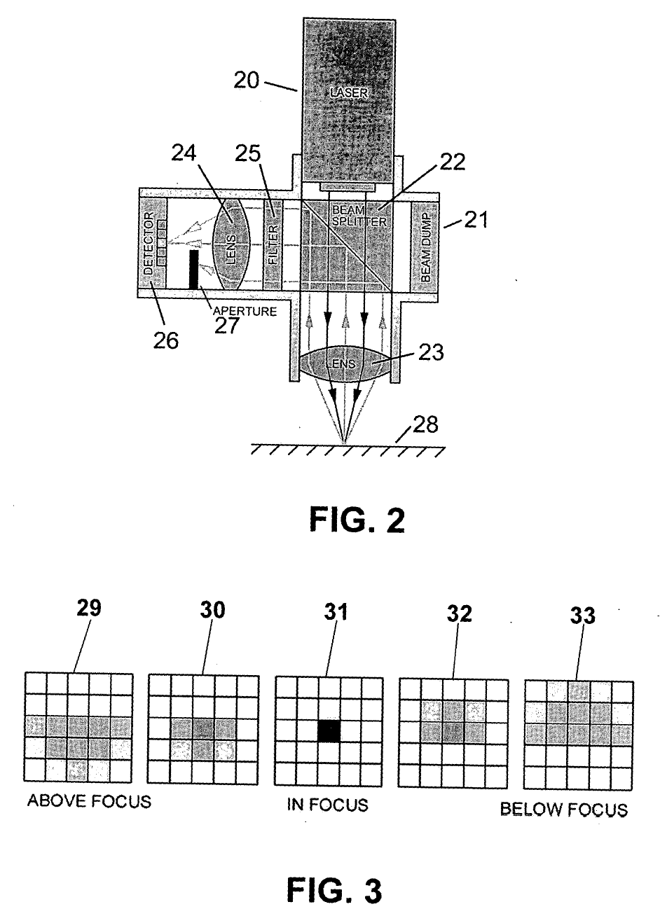

A laser distance measurement module 12 measures the distance between the focal point of its internal laser and the surface directly below it. The requirements of the distance measurement system 12 is that it should be able to resolve distance changes of approximately 25 microns about its operating range (when the focal point is slightly above or slightly below the surface), as well as it having the ability to detect when the focal point of the laser 20 is far above or far below the surface. Distance resolution away from the operating point is not crucial. In an exemplary laser distance measurement module 12 shown in FIG. 2, the module 2 includes a low-power, modulated laser 20, cubic beam splitter 22, short focal-length lenses 23 and 24, optical filter 25, aperture stop (knife edge) 27, 25-element photodiode detector array 26, and beam dump 21. Half of the laser beam passes through beam splitter 22 and is focused to a 100-micron diameter spot at the focal point of lens 23. The other half of the beam is captured by beam dump 21. When the focal point of the laser beam is coincident with surface 28, reflected light rays leave this spot in all possible trajectories. The light rays incident on lens 23 form a collimated beam prior to entering beam splitter 22. Half of this beam is reflected 90 degrees, through filter 25 and towards lens 24. This beam is then focused, illuminating the central pixel on detector array 26. Aperture 27 removes specific light rays to facilitate sign determination. The distance between lens 24 and detector array 26 is chosen so that a point source at the focal point of lens 23 is focused to a point at the plane of the detector array. A benefit of this distance measurement system is that it is not sensitive to the reflectivity of the surface.

When the focal point of the laser 20 is not coincident with surface 28, two effects cause the size of the spot on detector array 26 to grow. The first effect is simply that the laser's spot size on the surface is larger because it is out of focus. The second is that this spot is again out of focus when imaged onto the detector array. Both these effects enlarge the size of the spot, illuminating additional pixels as shown in the detector arrays illustrated in FIG. 3, in which detector array 31 illustrates the pixel intensity for the in focus beam. The convergence angle of the light cone from the laser is much narrower than the reflected light cone that enters the optical system. Because of this, the unfocussed image of the laser spot on the detector is the dominant effect responsible for the enlargement of the size of the image on the detector. A benefit of this effect is that the distance measurement system is not sensitive to the incident angle of the laser beam. The image on the detector closely resembles a circle under all normal operating conditions. Since the image diameter grows whether the focal point is above or below the surface, a method is required to determine the sign of the measurement. Sign determination is accomplished through the effect of aperture 27. Aperture stop (knife edge) 27 removes specific light rays so that no light will reach either the top or the bottom half-plane of the detector, depending on whether the focal point is above or below the surface (FIG. 3). When the focal point of lens 23 is above surface 28, the reflected light rays destined for the detector array converge prior to the plane of the detector. The light rays from the upper half of lens 24 are incident on the lower pixels of detector 26, but the light rays from the lower half of lens 24 are blocked by aperture 27 and never reach the upper pixels of detector 26. As a result, the pixel intensities on of the upper half-plane of detector array 29 and 30 are small compared to the lower half-plane pixel intensities. The reverse situation occurs when the focal point of lens 23 is below surface 28; the pixel intensities on lower half-plane of detector 32 and 33 are small compared to the upper half-plane. Using standard optics equations, software module 15 calculates distance based on the size of the spot, the difference in intensities between the upper and lower half-planes, and the geometry of the optical system.

Each pixel in detector array 26 is a low-noise precision photodiode. These photodiodes are reverse-biased with 5V to improve the range over which the photodiode current is linear with optical power. The current from each photodiode is used to drive an operational amplifier operating as a transimpedance amplifier. The transimpedance amplifiers output voltages proportional to the pixel light intensity. Left unchecked, the signal-to-noise ratio (SNR) of the image on the detector would be extremely poor because the laser light is reflected off various surfaces in an environment containing all sorts of stray light. Various methods may be used to remove the background and improve the SNR (especially the light from an incorporated surgical laser), for example using a filter 25 to filter the light prior to the detector. Filter 25 is designed to highly attenuate light at the wavelength of the surgical laser while minimally attenuating light at the wavelength of the distance signal. In another SNR improved method, laser 20 is modulated with a square-wave and then the transimpedance amplifier outputs from detector 26 are run though lock-in amplifiers so only the modulated reflected signal remains. The lock-in amplifiers act like band-pass filters perfectly centered about the modulation frequency followed by low pass filters. The signals out of the lock-in amplifiers are DC voltages proportional to the signal intensities at the various pixels but unaffected by ambient light levels. These signal are then digitized by an analog-to-digital converter. Software module 15 running onboard the robot main controller has access to the pixel intensity readings through electrical interface 19. This software module analyzes the pixel intensity as a function of radial distance from the central pixel to calculate a spot size and analyzes the central column to determine the sign of the distance measurement. From the spot size and sign, a simple geometric optics calculation is performed in software to determine the distance between the focal point and the surface. Softwared module 15 may be run on a microcontroller, general purpose computer or may be a hard wired device.

Many permutations to the distance measurement system 12 could be made and still serve the primary function of measuring focal point-to-surface distance. Permutations include but are not limited to: increasing the number of pixels in the detector array or using a CCD camera element as the detector to improve distance resolution; adding additional lenses or arranging the existing ones to adjust the rate the image size grows with changes in focal point-to-surface distance; including intelligent amplifiers to dynamically adjust gain or subtract offset to improve the range of pixel intensities that the system can digitize; and employing more complex algorithms to calculate spot size that are robust on certain biological materials such as ones that are semi-transparent, or increase robustness in the presence of smoke, blood or water.

Tactile feedback is rendered through haptic hand control 18 at the surgical robot workstation. The desired feedback force vector in such hand controls is set with high-level commands from the host computer (main control system 14), and a feedback loop internal to the hand control 18 adjusts actuator current to produce the desired feedback forces. Thus it is sufficient to describe how the desired feedback forces are calculated based on the distance measurement and robot kinematics because the art of actually rendering such forces is well known in the field of haptics.

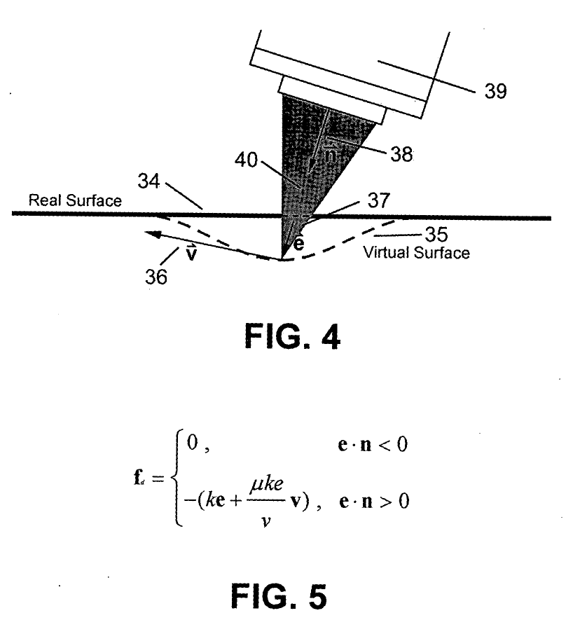

The nomenclature used to describe the force feedback-rendering algorithm is shown in FIG. 4. When describing how the force feedback is rendered, it is convenient to imagine virtual surface 35 initially coincident with real surface 34 but deformable by laser beam 40 from the distance measurement module 12. Feedback forces are synthesized in response to these deformations and the velocity of a point fixed on the laser beam. In the case where the laser is well above the surface, no feedback forces are generated by software module 15. The motion of the laser is unimpeded. The TFLS is dormant in this regime, and the motion of the robot arm 17 is controlled in the regular fashion as though the TFLS was absent.

As shown in FIG. 4, define n as unit vector 38 along the axis of laser beam 40, define e as vector 37 coaxial with n starting at the real surface and terminating at a point on the laser beam (close to, but not necessarily equal to, the focal point), and define v as vector 36 originating from the termination point of e and having magnitude and direction equal to the point's velocity. The optical distance measurement system therefore provides a measurement of the dot product of e and n. The direction of n can be found using knowledge of the robot arm joint coordinates (available a priori to main control system 14) and standard robot kinematics. Similarly, the magnitude and direction of v can be found from velocity and position data from the joint encoders (available a priori to main control system 14) using standard robot kinematics. The mathematical expression for the desired feedback force, f_{d}, is given in FIG. 5. In this figure, k and μ are respectively the spring constant and the damping (or frictional) coefficient of the virtual surface. This desired feedback force is then sent from main control system 14 to haptic hand control 18 for rendering.

It is instructive to examine the haptic effect of the various terms in the equation given in FIG. 5. In the case where the laser 20 is far above the surface, e and n will have opposite directions and thus the dot product will be negative. No feedback forces will be generated and the robot arm 17 will move freely. As the laser 20 nears the surface, e and n will have the same direction and force feedback will be generated. The first term ke is a spring force and is needed to create the sensation of a solid surface. The second term μkev/v is a Coulomb-like surface friction where ke takes the place of the normal force. The damping term applies friction in all directions of motion when the laser's focal point is coincident with the surface. This results in a familiar feel; when a conventional tool touches a surface there is friction to move it laterally across that surface. The damping term accomplishes the same effect, although only light ever contacts the surface. When the dot product of e and n is very close to zero, the desired force alternates rapidly between no friction and friction, resulting in familiar stick-slip behavior.

Many permutations of the force-feedback rendering algorithm could be made and still serve the primary function of providing a sense of touch with light. This could include but is not limited to the following: nonlinear terms in the equation given in FIG. 5 to make the applied force vary non-linearly with distance, terms proportional to the integral or derivates of e, or additional expressions in the piecewise formula. In addition, a remote distance measuring system could use, instead of laser 20, a sonar device, an incoherent light source, or other forms of non-damaging electromagnetic radiation or sound waves. The distance measuring system 12 disclosed here uses a system based on the focus size of a beam. However, the distance measuring system 12 could use other techniques for the remote measurement of distance with electromagnetic radiation or sound waves.

Surgical laser module 13 (FIG. 1) may be a separate surgical laser focused at the same spot as the distance measurement laser, with nearly parallel trajectories. This module also includes standard control electronics (such as a closed-loop current controller) so that the desired laser intensity can be set by commands from main control system 14. A benefit of having a separate focus laser is that various cutting laser diodes with different properties for various biological materials can be interchanged without having to recalibrate the distance measurement system 12. Numerous permutations exist for the integration of the surgical laser 13. The surgical cutting laser may be the same laser as the laser used to acquire the distance signal. In this case, when no cutting power is desired, the surgical laser output power is extremely low so that only enough optical power is used to operate the distance measurement system. The gains of the detector array pixel amplifiers are variable so that the output does not saturate when the laser is used for cutting. The surgical laser 13 may also be a separate laser from the one used to acquire the distance signal but share a common optical path and focused at the same point as the distance-measurement laser 12.

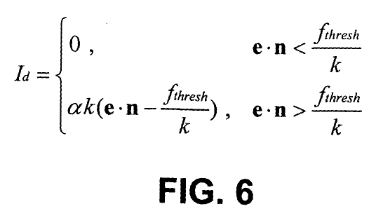

Software module 16 may be used for controlling surgical cutting laser intensity. In this embodiment, the intensity of the surgical laser is controlled by applied force. With a knife, increasing force results in deeper and faster cuts. With a haptic laser, an analogous situation is possible. Since the desired force feedback along the axis of the laser is proportional to the dot product of e and n, it is possible to use this term to also control laser intensity. Electronic circuits for closed-loop current control of laser intensity is well understood and thus software module 16 can set surgical laser intensity with a single function call. Thus it is sufficient to specify the desired laser intensity as a function of the parameters available to the software module, namely the distance measurement. The desired intensity is given by the piecewise equation in FIG. 6. This equation specifies that the laser intensity should be zero until the force feedback reaches a threshold level, at which point the intensity should increase linearly with applied force. Incorporation of the surface spring constant into this equation makes the units for the two new constants convenient: α has units of power per unit force and relates surgical laser output power to applied force; fₜₕᵣₑₛₕ has units of force and specifies the threshold force below which the surgical laser is not active.

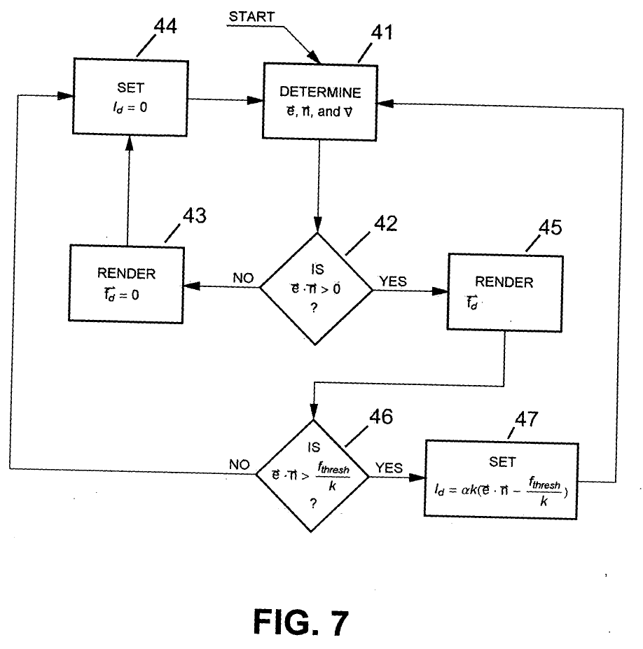

The flow diagram for the operations carried out by this software module and the laser intensity software module is given in FIG. 7 (effect of both modules shown together). The logic follows largely from the piecewise equations given in FIGS. 5 and 6. The loop runs at a frequency close to 1 kHz so that haptic “jitter” is not detectable by the human operator. Thus, in step 41, determine e, n and v. In step 42, check whether the dot product of e and n is greater than zero. If yes, render fd in step 45 according to the equation in FIG. 5, and if the dot product of e and n is greater than fₜₕᵣₑₛₕ (step 46) then render Id according to the equation in FIG. 6 (step 47). If the decision in step 42 gives the result no, then set fd equal to zero in step 43, set Id to zero in step 44 and return to start.

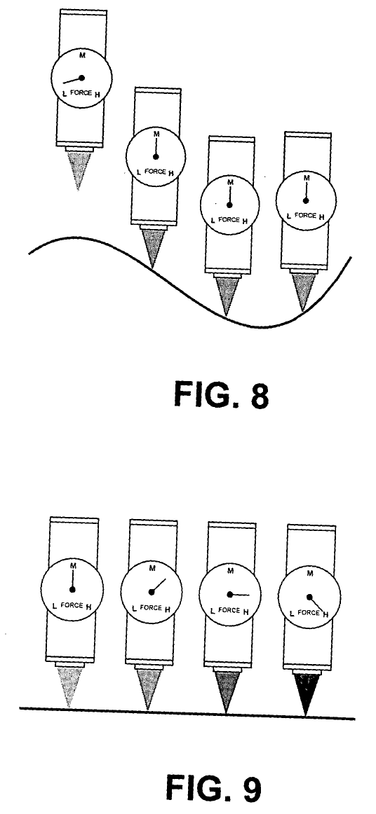

In a method of use of the TFLS system, the surgeon will direct the laser towards the surface using the standard hand control for moving the robot's end-effector. As the focal point of the laser nears a surface, the surgeon will feel an opposing force that rapidly increases wheri the focal point of the laser is coincident with the surface. The surgeon will experience the sensation of a hard surface although nothing but light will actually be touching it. If the surgeon maintains constant force and moves the laser laterally, the laser will track the profile of the surface as shown in FIG. 8. In FIG. 8, the TFLS is represented as a force sensor and laser beam, and the force and distance to the surface is kept constant across the surface. Once the focal point of the laser is coincident with the surface, additional downwards force will increase the cutting power of a surgical laser focused at the same spot as shown in FIG. 9, where, as the force increases, the laser intensity, illustrated by shading, increases. This is not unlike how a knife responds to increasing downwards force. The laser system may be used as a surgical laser for cutting and ablation, but with the surgical laser disabled, it may still be a useful tool. It will provide the remote surgeon with a non-contact way to feel around the surgical site, which could be particularly important given the limited depth perception offered by microscopes, especially during remote or robot procedures. The trajectory of the laser will also appear in the surgical microscope and on the desk-mounted displays, providing the last of the missing sensory cues. It is possible to calculate laser trajectory because the robot controller monitors robot joint angles. From a priori laser beam geometry the control system 14 can infer the trajectory and digitally create a phantom image to overlay on the true image of the surgical field.

Many permutations of the laser intensity control algorithm could be made and still serve the primary function of controlling laser intensity by operator applied force. This could include but are not limited to the following: adding other terms to the expressions of the piecewise equation or adding new expressions, and incorporating a force sensor in the workstation hand controller to directly sense applied force along the laser beam axis (in the hand controller coordinate system) and running the intensity control algorithm form this information.

In further embodiment, the TFLS may incorporate a semi-passive robot arm. In this embodiment, the TFLS is similar to the first embodiment except the surgeon directly manipulates the robot arm with a hand grip mounted directly on the robot arm. When the laser is far from a surface, the robot arm is passive in the sense that the operator can move the arm relatively freely. When the laser's focal point nears the surface, the control system 14 adjusts the algorithm to produce feedback forces and create the sensation of a false surface in the same method as the first embodiment, of course with the exception that the forces are rendered at the robot arm instead of at a separate hand control. The actuator for the robot arm in this embodiment is a part of the robot arm. Synthesizing forces is well understood and can be accomplished with closed-loop control of actuator current to produce the correct actuator forces/torques and robot statics to relate actuator forces/torques to the feedback force at the end-effector.

In a still further embodiment, the TFLS is integrated as a non-contact light probe. In this embodiment, the TFLS is indistinguishable from the first two embodiments with the absence of the surgical cutting laser and associated laser intensity control algorithm. The use of the device is a light probe, providing the remote surgeon a non-contact method of “feeling” around the surgical sight to gauge distances and surface profiles. This is of importance to the remote. surgeon given the limited depth perception of surgical microscopes and display monitors. It is also conceivable to employ this light probe with a conventional surgical laser to provide the sense of touch, while allowing the surgeon to retain the ability to adjust laser intensity using a conventional method such as turning a knob.

The laser distance measurement module provides a measurement of the focal point-to-surface distance regardless of surface reflectivity, incident angle of the laser beam, or ambient light levels. The slope of the target surfaces affects the distance measurement to some degree (slope error); however, they do not cause an error in the zero distance reading (no offset error). At zero distance the scattered light rays are guaranteed by design to hit only the central pixel regardless of incident angle or media (transparent and semitransparent media are not considered). Varying incident angle and media may cause slope and offset errors. Moderate slope-type distance errors are acceptable because they affect only the apparent compliance of the virtual surface and the operator may find this additional haptic information useful. Offset errors are not as acceptable because the onset of haptic feedback will not occur at a fixed distance, resulting in chatter and poor haptic feedback. The coefficients in the control algorithm, k and μ, may be dynamically adjusted based on the detector array pixel voltages to relay additional haptic information to the operator. A sensible way to adjust these parameters using information already available may make the surface feel softer with more friction if less light is scattered back to the detector. When less light is detected, it suggests that more light is absorbed by the surface. When incorporated with a surgical laser it would be this absorbed laser light that performs the cutting. Hence the compliance and friction would relate (albeit loosely) to how quickly the surface would cut. Although these haptic sensations would not necessarily correlate with what one would feel when dragging a tool across the surface, they are potentially more useful to the operator. Since it would be laser light doing the cutting, the properties that describe the interaction of laser light with the surface should be communicated to the operator.

For use as an FDA-approved surgical laser system operating with a surgical robot such as neuroArm™, the design of the distance measuring module, including the optical path and the algorithm used, should be optimized and characterized through a computer model of the module and tested on a wide range of surfaces, and then tested for use by surgeons with a view to synthesizing haptic feedback that increases operator comfort, performance, and acceptance of laser technology. Additional sensor readings or auxiliary information may also improve performance of the TFLS.

Immaterial modifications may be made to the embodiments of the invention described here without departing from the invention.

What is claimed is:

1. A tactile feedback system, comprising:
a robot arm;
a remote distance measuring device mounted on the robot arm, the remote distance measuring device having an output corresponding to a distance measure;
a hand control for the robot arm; and
a force feedback control system responsive to the distance measure to control force applied to the hand control.

2. The tactile feedback system of claim 1 further comprising an actuator for the hand control; and the force applied to the hand control is applied by an actuator attached to the hand control.

3. The tactile feedback system of claim 1 further comprising a cutting laser mounted on the robot arm.

4. The tactile feedback system of claim 3 in which the cutting laser is a surgical laser.

5. The tactile feedback system of claim 3 in which the force feedback control system is configured to adjust cutting laser power output depending on the distance measure.

6. The tactile feedback system of claim 1 in which the force feedback control system is configured to apply a force to the hand control that depends on the motion of the robot arm.

7. The tactile feedback system of claim 3 in which the remote distance measuring device comprises a second laser.

8. The tactile feedback system of claim 7 in which the remote distance measuring device is configured to determine distance based on spot size of a beam emitted by the second laser and incident on a surface.

9. The tactile feedback system of claim 1 in which the hand control is physically remote from the robot arm.

10. The tactile feedback system of claim 1 in which the control system is configured to adjust cutting laser power output depending on the distance measure.

11. The tactile feedback system of claim 4 in which the remote distance measuring device comprises a second laser.

12. The tactile feedback system of claim 11 in which the cutting laser and second laser are oriented on the robot arm to have coincident focal points of laser beams emitted by the first laser and second laser.

13. The tactile feedback system of claim 1 in which the controller is configured to apply a force to the hand control that varies non-linearly with the distance measure.
